(19) United States
(12) Patent Application Publication
(10) Pub. No.: US 2007/0225492 A1
(43) Pub. Date: Sep. 27, 2007

(54) 3,6-Diazabicyclo[3.1.1]Heptane Derivatives with Analgesic Activity

(76) Inventors: Gerard Aime Pinna, Sassari (IT); Giovanni Loriga, Sassari (IT); Giorgio Cignarella, Milano (IT)

Correspondence Address: YOUNG & THOMPSON, 745 SOUTH 23RD STREET, 2ND FLOOR, ARLINGTON, VA, 22202, US

(21) Appl. No.: 11/596,083

(22) PCT Filed: May 9, 2005
(86) PCT No.: PCT/EP05/04994

§ 371 Date: Jan. 4, 2007

(30) Foreign Application Priority Data
May 12, 2004 (IT) MI2004A000954

(51) Int. Cl. C07D 487/08

(52) U.S. Cl. 540/347

(57) ABSTRACT

The invention relates to compounds of general formula (I), wherein R and R₁, different from one another, are: a C₂-C₈ straight or branched acyl group; and a group of formula (II), wherein B and R₂ are as defined in the description. The compounds (I) have higher central analgesic activity than morphine and are substantially free from the side effects of morphine or other central analgesics. The invention further relates to a process for the preparation of the compounds (I).

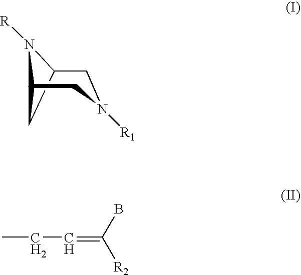

### FIELD OF THE INVENTION

The present invention relates to 3,6-diazabicyclo[3.1.1]heptane derivatives, the use thereof as agents with central analgesic activity in the preparation of medicaments and pharmaceutical compositions containing them.

### BACKGROUND OF THE INVENTION

Morphine-like opioids are substances having central analgesic activity showing, like morphine, marked selectivity towards opioid receptors μ, δ and κ. To date, efforts of the pharmaceutical chemistry were mainly focused on the development of central analgesics with maximum selectivity towards receptor μ, which mediates analgesia. However, a number of synthetic central analgesics also act on other opioid receptors, namely receptors δ and κ, whose stimulation induces the undesired side effects of this class of medicaments. Therefore, there is still the need for substances with analgesic activity which overcome said drawbacks.

WO 9523152 and WO 9847902 in the Applicant's name disclose 3,8-diazabicyclo[3.2.1]octane derivatives and 3,9-diazabicyclo[3.3.1]nonane derivatives which induce less tolerance than morphine.

### DETAILED DISCLOSURE OF THE INVENTION

The present invention relates to compounds of general formula (I)

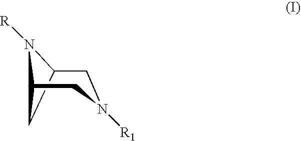

wherein R and R₁, which are different from one another, are:
a straight or branched C₂-C₈ acyl group; or
a group selected from:

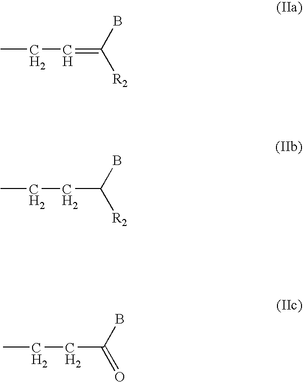

wherein B is:
a C₆-C₁₀ aryl group, optionally substituted with one or more groups, which can be the same or different, selected from C₁-C₃ alkoxy, C₁-C₂ haloalkyl, C₁-C₃ alkyl, halogens, carboxy, cyano, nitro, CONHR₃, wherein R₃ is straight or branched C₁-C₄ alkyl;
a C₅-C₇ cycloalkyl group;
a 5 or 6 membered aromatic heterocycle, optionally benzofused, containing at least one heteroatom selected from nitrogen, oxygen, sulfur; said heterocyclic group optionally bearing one or more substituents among those indicated for the aryl group;

and in which R₂ is hydrogen, straight or branched C₁-C₄ alkyl, a C₅-C₇ cycloalkyl group or phenyl optionally substituted with one or more groups, which can be the same or different, selected from those indicated above for the aryl group;

and the pharmaceutically acceptable salts thereof.

C₂-C₈ Acyl groups are preferably acetyl, propionyl, butyryl, isobutyryl, valeryl, isovaleryl, pivaloyl, caproyl.

Aromatic heterocycles are preferably pyrrole, furan, thiophene, imidazole, oxazole, thiazole, pyridine, pyrimidine, pyridazine, pyrazine, benzothienyl.

Pharmaceutically acceptable salts are those with halo acids, such as hydrochloric acid, hydrobromic acid; mineral acids, such as sulfuric and phosphoric acid; organic acids, such as acetic, propionic, succinic, glutaric, fumaric, benzoic, salicylic. In case a carboxylic group is present in the compounds of formula (I), it can be in the salified form with alkali or alkaline-earth metal bases, such as sodium, potassium, calcium, magnesium; non toxic metal bases; non toxic organic amines.

A first preferred group of compounds of formula (I) consists of compounds (IA) 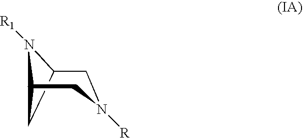
in which R is C₂-C₈ acyl as defined above and R₁ is a group of formula (IIa-c) as defined above.

A second preferred group of compounds of formula (I) consists of compounds (IB) 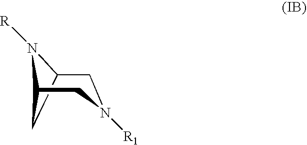
in which R is C₂-C₈ acyl as defined above and R₁ is a group of formula (IIa-c) as defined above.

Most preferred are the compounds of formula (IA) and (IB) in which the groups R and R₁ are respectively acetyl or propionyl, most preferably propionyl and a group of formula (IIa), (IIb) or (IIc) in which B is phenyl, optionally substituted as defined above, and R₂ is hydrogen or C₁-C₄ alkyl, preferably methyl or ethyl.

Particularly preferred are the following compounds:
3-propionyl-6-cinnamyl-3,6-diazabicyclo[3.1.1]heptane (IAa);
3-propionyl-6-[(2E)-3-(2′-chlorophenyl)-prop-2-enyl]-3,6-diazabicyclo[3.1.1]heptane (IAb);
3-propionyl-6-[(2E)-3-(3′-chlorophenyl)-prop-2-enyl]-3,6-diazabicyclo[3.1.1]heptane (IAc);
3-propionyl-6-[(2E)-3-(4′-chlorophenyl)-prop-2-enyl]-3,6-diazabicyclo[3.1.1]heptane (IAd);
3-propionyl-6-[(2E)-3-phenyl-but-2-enyl]-3,6-diazabicyclo[3.1.1]heptane (IAe);
3-propionyl-6-[(2E)-3-(4′-chlorophenyl)-but-2-enyl]-3,6-diazabicyclo[3.1.1]heptane (IAf);
3-propionyl-6-[(2E)-3-(3′,4′-dichlorophenyl)-but-2-enyl]-3,6-diazabicyclo[3.1.1]heptane (IAg);
3-propionyl-6-[(2E)-3-phenyl-pent-2-enyl]-3,6-diazabicyclo[3.1.1]heptane (IAh);
3-propionyl-6-[(2E)-3-(4′-nitrophenyl)-prop-2-enyl]-3,6-diazabicyclo[3.1.1]heptane (IAi);
3-propionyl-6-(3-phenylpropyl)-3,6-diazabicyclo[3.1.1]heptane (IAl);
3-propionyl-6-[3-(4′-nitrophenyl)propyl)]-3,6-diazabicyclo[3.1.1]heptane (IAm);
3-propionyl-6-[3-(4′-chlorophenyl)propyl)]-3,6-diazabicyclo[3.1.1]heptane (IAn);
3-propionyl-6-(3-phenylpropyl-3-one)-3,6-diazabicyclo[3.1.1]heptane (IAo);
3-propionyl-6-[3-(4′-chlorophenyl)propyl-3-one]-3,6-diazabicyclo[3.1.1]heptane (IAp);
3-cinnamyl-6-propionyl-3,6-diazabicyclo[3.1.1]heptane (IBa).

The invention further relates to a process for the preparation of compounds (I).

The compounds of formula (IA) can be prepared by reaction of a compound of formula (IIIA) or (IIIB)

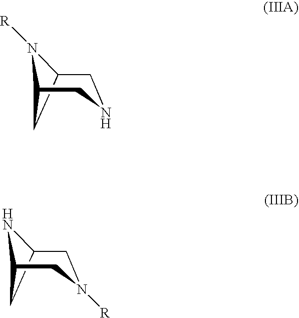

in which R is a C₂-C₈ acyl group as defined above,

with a compound of formula (IVa)-(IVc)

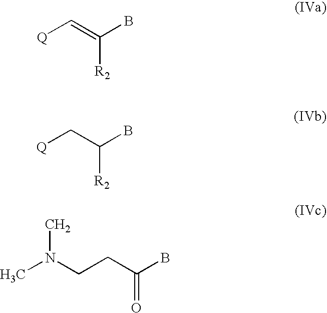

wherein R₂ and B are as defined above and Q is a CHO group or a CH₂X group in which X is a leaving group, preferably selected from halogen, mesyl and tosyl.

The reaction between the compounds (IIIA) or (IIIB) and the compounds of formula (IVa-c) is carried out according to conventional methods, known to those skilled in the art. The reagents are usually in stoichiometric or slightly different ratios, depending on the reactivity of the specific reagent. It should be pointed out that the compounds (IA) can also be obtained starting from the compounds of formula (IIIA), since in the course of the reaction migration of the acyl group to give compounds (IIIB) occurs; this rearrangement is also observed in the homologous diazabicyclooctanes series (Tetrahedron, 1963, 19, 143-148).

The compounds of formula (IVa)-(IVc) are known or can be prepared with conventional methods. Compounds (IVa) can be prepared by reduction of substituted acrylic acids or esters thereof with metal hydrides and subsequent conversion of the resulting alcohols to halides or aldehydes (IV), for example according to what illustrated in Scheme 1a, in which B, R₂ and Q are as defined above.

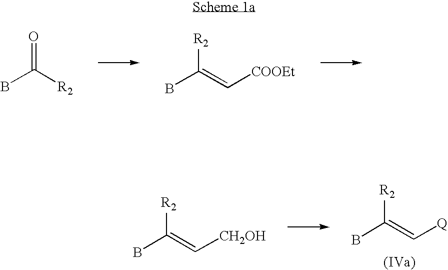

Compounds (IVb) can be prepared by reduction of the double bond of acrylic esters with hydroxylamine-O-sulfonic acid, followed by reduction of the ester group with a metal hydride and subsequent conversion of the resulting alcohol to bromide with PBr₃, as illustrated in scheme 1b:

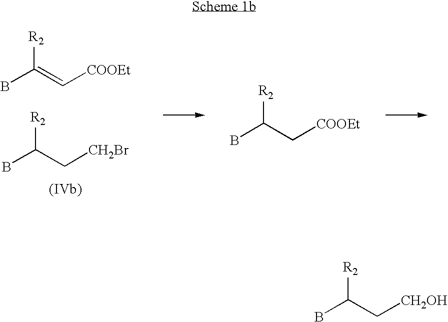

Compounds (IVc) can be prepared by conversion of an acetyl derivative to the corresponding Mannich bases with 37% formaldehyde and dimethylamine (scheme 1c).

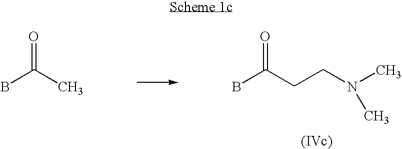

The compounds of formula (IIIA) and (IIIB) can be obtained by acylation of a compound of formula (VA) or (VB)

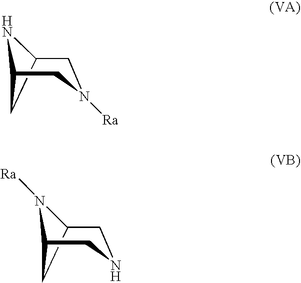

in which Ra is an amino-protecting group which can be removed by hydrogenolysis, selected from benzyl or benzyl substituted with a methoxy group, for example 4-methoxy-benzyl (MPM) or 3,4-dimethoxy-benzyl (DMPM), and subsequent removal of the protective group. According to a particularly preferred embodiment of the invention the protective group is benzyl.

The acylation reaction is usually carried out with acid chlorides in an inert reaction medium, such as a linear or cyclic ether, a ketone, an optionally halogenated hydrocarbon. The presence of a proton acceptor, for example a tertiary amine, is preferred. Alternatively, the acylating agent can be a carboxylic acid anhydride.

The compounds (VB) can in turn be obtained by introducing in a compound (VA) a protective group Ra′, namely an amino-protecting group which can be removed by hydrolysis, under acid or basic conditions. Said group is preferably selected from t-butoxycarbonyl (BOC), fluorenylmethoxycarbonyl (FMOC), vinyloxycarbonyl (VOC), allyloxycarbonyl (ALOC) and trichloroethoxycarbonyl (TROC). According to a preferred embodiment of the invention, the protective group is BOC. Selective removal of the protective group Ra affords the compounds (VB).

The compounds (VB) are key intermediates for the preparation of the compounds of the invention of formula (IB). To this purpose, the compounds (VB) are reacted with a compound of formula (IV) as defined above, to give a compound of formula (VIII)

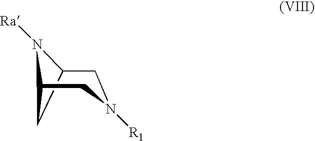

in which Ra′ is as defined above and R₁ is a group of formula (II).

The compound (VIII) is subjected to hydrolysis under acid or basic conditions, depending on the protective group, to give compound (IX)

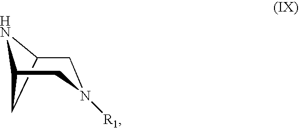

which is acylated to give the compounds (IB).

Scheme 2 graphically resumes what explained above.

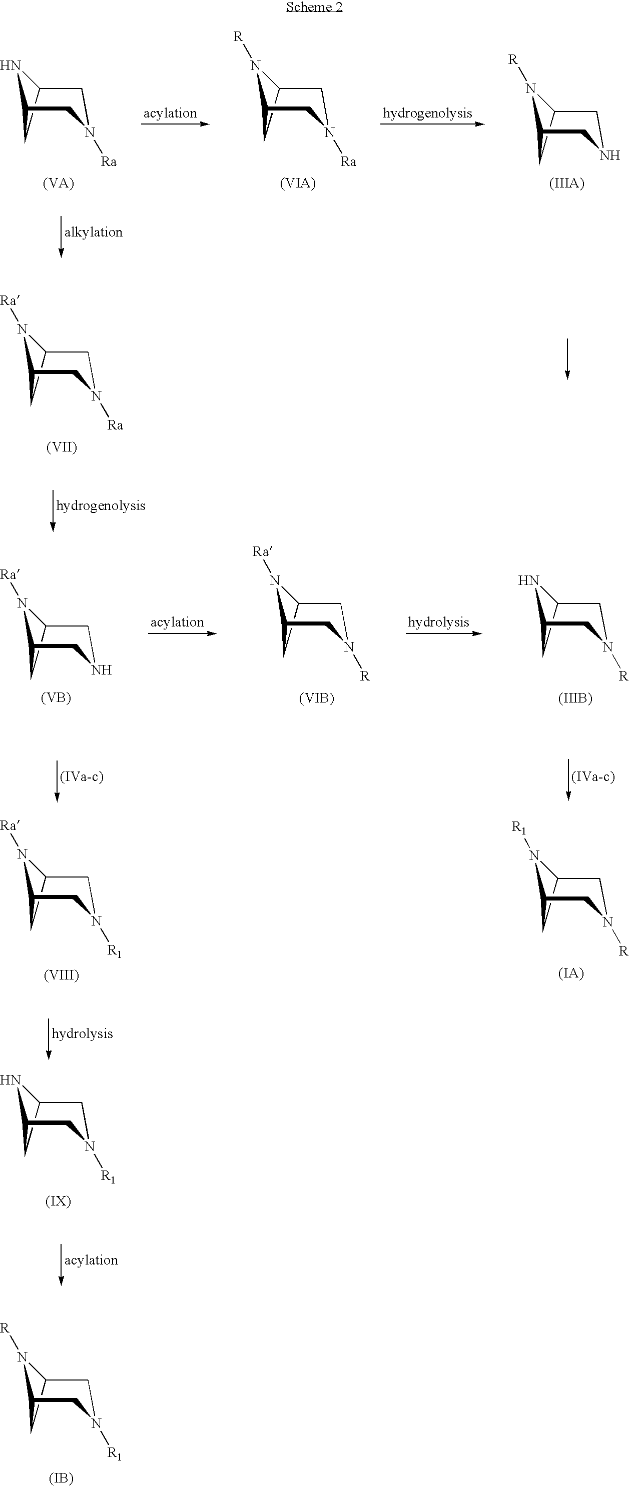

The preparation of the compounds of formula (VA) is illustrated in Scheme 3. Glutaryl-dichloride is brominated to give meso-dimethyl-α,α′-dibromoglutarate (X), which is condensed with an amine RaNH₂, in which Ra is as defined above, to give azetidine (XI) as an isomeric mixture. The cis isomer is subjected to monoaminolysis by treatment with the same amine RaNH₂ to give the amide (XII). Reduction of the amide affords alcohol (XIII), which is transformed into mesylate (XIV) and cyclized to give compound (XV). Compound (XV) is subjected to catalytic hydrogenation to give compound (XVI), which is reduced with hydrides to give (VA).

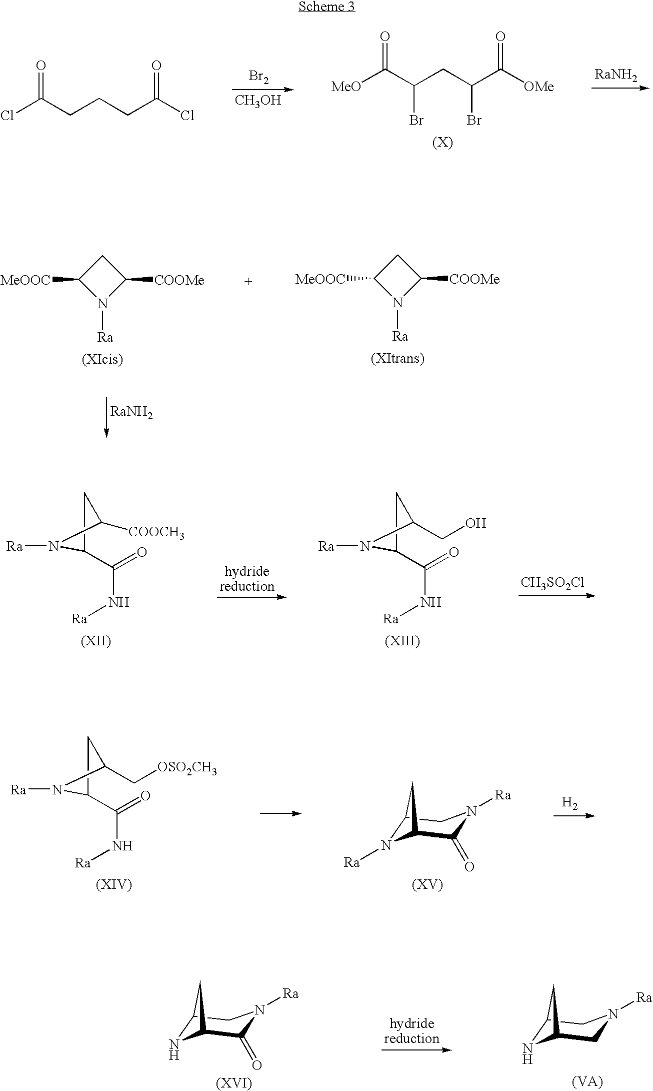

Scheme 4 shows in particular the synthesis of a compound (VA) in which Ra is benzyl, and the synthesis of a compound (VB) in which Ra′ is t-BOC starting from (VA).

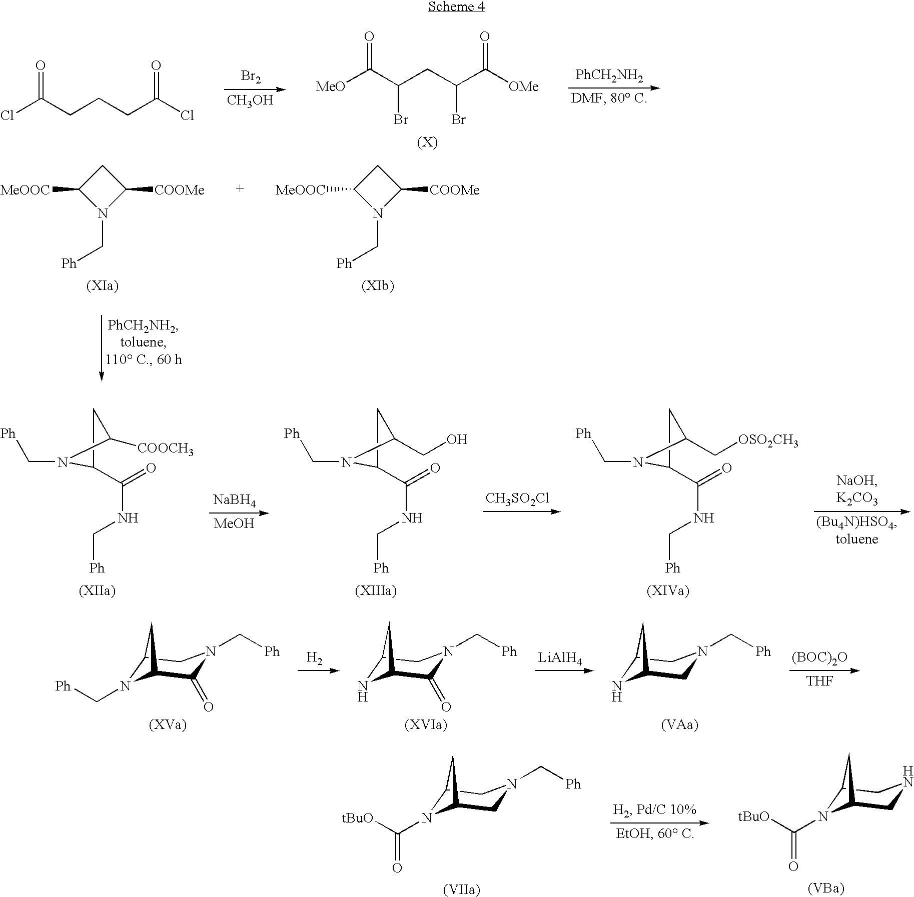

The compounds of formula (I) have central analgesic activity and proved more potent than morphine and the compounds disclosed in WO 9523152 and WO 9847902; moreover, they do not generally induce abstinence and they cause less tolerance or dependence than morphine after chronic treatment, by virtue of their high selectivity towards μ receptors (as shown in the following table 2).

“They do not generally induce abstinence” means that they have an activity from 3 to 20 times lower than morphine in the jumping test in the mouse, after chronic administration of equivalent analgesic doses three times a day for seven consecutive days.

It is therefore object of the present invention the use of the compounds of formula (I) for the preparation of a medicament which induces analgesia in the central nervous system of a mammal, in particular man, in the need of analgesic treatment.

For the envisaged therapeutic uses, the compounds (I) or salts thereof will be formulated in a therapeutically effective amount in suitable pharmaceutical compositions according to conventional techniques and excipients, such as those described in “Remington's Pharmaceutical Sciences Handbook” XVII Ed. Mack Pub., N.Y., U.S.A.

Examples of pharmaceutical compositions are tablets, capsules, granulates, soluble powders, drops, elisir, syrups, injectable forms, suppositories.

The dosages and the posology will be determined by the physician according to the severity of the disease, patient's conditions and possible interactions with other medicaments.

The following examples further illustrate the invention.

### EXAMPLES

### Example 1—Methyl 2,4-dibromo Glutarate (X)

Glutaryl chloride (20.00 g, 118.33 mmoles) was added with 13.33 ml (260.30 mmoles) of bromine at a temperature of 90° C. and the solution was irradiated with a 300 W lamp for 4 hours. The mixture was cooled to room temperature and 71.89 ml (1775 mmoles) of dry methanol was added with cooling (ice bath), then stirred for 12 hours. The solution was concentrated and the brown oily residue was added with 72 ml of water. The aqueous solution was repeatedly extracted with ethyl ether which, washed in succession with 5% NaHCO₃ and 2% NaHSO₃, dried (Na₂SO₄) and concentrated to give 35.71 g of an orange oil. The crude oil was purified by distillation in a bulb tube at 140-145° C./0.1 mmHg.

Yield: 95%

R_{f}: 0.56 (6:1 hexane-ethyl acetate)

B.p.: 145° C./0.1 mmHg (Lit.²¹: 120° C./0.01 mmHg)

IR ν (cm⁻¹): (Film) 1730 (C═OR).

¹H-NMR (CDCl₃): 2.49-3.00 (m, 2H, CH₂), 3.82 (s, 6H, CH₃x2), 4.36-4.50 (m, 2H, CHx2).

### Example 2-trans-Methyl 1-benzylazetidine-2,4-dicarboxylate (XIa trans) and cis-methyl-1-benzylazetidine-2,4-dicarboxylate (XIa cis)

A solution of dibromoglutarate (X) (35.50 g, 111.64 mmoles) and benzylamine (36.60 ml, 334.92 mmoles), in 170 ml of dimethylformamide was left under stirring for 4 hours at 80° C. The solvent was evaporated off and the residue was dissolved in dichloromethane. The solution washed with a saturated NaHCO₃ solution, dried (Na₂SO₄) and concentrated to give 42 g of an oily residue. The crude oil showed two main TLC spots (8:2 petroleum ether-ethyl acetate) with R_{f} of 0.43 and 0.26. The two isomers were separated by flash chromatography (SiO₂), eluting with a 8:2 petroleum ether-ethyl acetate mixture to give a fraction which, after evaporation, afforded 6.77 g of trans-XI.

Yield: 18%

R_{f}: 0.43 (8:2 petroleum ether-ethyl acetate)

B.p.: 148° C./0.1 mmHg

IR ν (cm⁻¹): (Film) 1590 (C═C, Ar), 1730 (C═OR).

¹H-NMR (CDCl₃): 2.42-2.60 (m, 1H, CH), 3.64 (t, 2H, CHx2, J=4.0 Hz), 3.65 (s, 6H, CH₃×2), 3.87 (s, 2H, CH₂), 4.18-4.29 (m, 1H, CH), 7.09-7.47 (m, 5H, ArH).

Evaporation of the second fraction gave 17.30 g of cis-XI.

Yield: 48%

R_{f}: 0.26 (8:2 petroleum ether-ethyl acetate)

B.p.: 140° C./0.1 mmHg

IR ν (cm⁻¹): (Film) 1600 (C═C, Ar), 1720 (C═OR)

¹H-NMR (CDCl₃): 2.27-2.60 (m, 2H, CH₂), 3.60 (t, 2H, CHx2), 3.63 (s, 6H, CH₃x2, J=6.6 Hz), 3.88 (s, 2H, CH₂), 7.09-7.47 (m, 5H, ArH).

### Example 3-(1-Benzyl-4-benzylcarbamoyl-azetidin-2-yl)methyl acetate (XIIa)

A solution of (XI cis) (11.01 g, 41.81 mmoles) and benzylamine (4.56 ml, 41.81 mmoles) in toluene (56 ml) was refluxed for 60 hours. The solvent was evaporated off to give 15 g of a crude solid which was purified by flash chromatography (SiO₂) eluting with a 5:5 petroleum ether-ethyl acetate mixture to give 7.77 g of (XIIa) as a white solid.

Yield: 55%

R_{f}: 0.20 (7:3 petroleum ether-ethyl acetate)

m.p.: 94-96° C. (isopropyl ether)

IR ν (cm⁻¹): (Nujol) 1600 (C═C, Ar), 1670 (C═OR), 1730 (C═OR, ester)

UV λ max (log ε): 206.6 (4.23)

¹H-NMR (CDCl₃): 2.13-2.30 (m, 1H, CH), 2.68-2.82 (m, 1H, CH), 3.60-3.92 (m, 4H), 3.68 (s, 3H, CH₃), 4.15-4.38 (m, 2H, CH₂), 7.03-7.44 (m, 10H, ArH).

Anal.: for C₂₀H₂₂N₂O₃, Calc. (Found) C, 70.90; (70.80); H, 6.55; (70.80); N, 8.28; (8.16).

### Example 4—1-Benzyl-2-benzilamido-4-hydroxymethylazetidine (XIIIa)

A methanol solution (40 ml) of (XIIa) (4.00 g, 11.82 mmoles) was added, under stirring and at a temperature of 0° C., with sodium borohydride (1.35 g, 35.46 mmoles) in small portions. The mixture was reacted for 12 hours, added with water (40 ml), then concentrated. The resulting aqueous solution was extracted with dichloromethane, dried (Na₂SO₄) and evaporated to give 3.76 g of pure (XIIIa) as a white solid.

Yield: 99%

R_{f}: 0.12 (5:5 petroleum ether-ethyl acetate)

m.p.: 92-94° C. (hexane/ether petroleum)

IR ν (cm⁻¹): (Nujol) 1600 (C═C, Ar), 1640 (C═OR).

UV λ max (log ε): 207.8 (4.23)

¹H-NMR (CDCl₃): 1.80-2.18 (m, 1H, CH), 2.43-2.60 (m, 1H, CH), 3.30-3.48 (m, 2H, CHx2), 3.60-3.76 (m, 2H, CH₂), 4.15-4.39 (m, 4H), 7.08-7.32 (m, 10H, ArH)

Anal.: for C₁₉H₂₂N₂O₂. Calc. (Found) C, 73.52; (73.06); H, 7.14; (7.28); N, 9.03; (8.95).

### Example 5—2-(1-Benzyl-4-benzylamido-azetidinil)-ethyl alcohol, methanesulfonic ester (XIVa)

A dichloromethane solution (97 ml) of (XIIIa) (8.77 g, 28.28 mmoles) was added with triethylamine (11.82 ml, 84.84 mmoles). The solution was cooled to 0° C. (ice bath and salt), and mesyl chloride (2.84 ml, 36.76 mmoles) was added. The mixture was allowed to react at 0° C. for 2.5 hours and then added with water. The phases were separated and the aqueous one was extracted with dichloromethane. The organic phase was dried (Na₂SO₄) and concentrated to give 13 g of a brown oily residue, which was purified by flash chromatography (SiO₂) eluting with a 2:8 petroleum ether-ethyl acetate mixture, to give 9.52 g of a yellow oil.

Yield: 86%

R_{f}: 0.39 (8.5:1.5 petroleum ether-ethyl acetate)

B.p.: 155° C./0.1 mmHg

IR ν (cm⁻¹): (Film) 1170 (SO₂simm.), 1360 (SO₂ₐsimm.), 1600 (C═C, Ar); 1680 (C═OR).

UV λ max (log ε): 214.5 (4.35)

¹H-NMR (CDCl₃): 1.92-2.10 (m, 1H, CH), 2.53-2.70 (m, 1H, CH), 2.80 (s, 3H, CH₃), 3.49-3.83 (m, 2H, CHx2), 3.61 (d, A of AB, 1H, CH, J=12.2 Hz), 3.77 (d, B of AB, 1H, CH, J=12.2 Hz), 3.89-4.41 (m, 4H), 7.09-7.47 (m, 10H, ArH)

Anal.: for C₁₉H₂₂N₂O₂. Calc. (found) C, 61.83 (61.58); H, 6.23 (6.21); N, 7.21 (7.18); S 8.25 (8.22).

### Example 6—3,6-Dibenzyl-3,6-diazabicyclo[3.1.1]eptan-2-one (XVa)

A suspension of (XIVa) (3.97 g, 10.21 mmoles), finely powdered NaOH (1.42 g, 35.73 mmoles), K₂CO₃ (2.82 g, 20.42 mmoles) and (Bu₄N)HSO₄ (0.34 g, 1.02 mmoles) in toluene (48 ml) was refluxed for four hours, then cooled to room temperature. The mixture washed with water and the aqueous phase was extracted with ethyl ether. The combined organic phases were dried (Na₂SO₄) and the solvent was evaporated off to give 4.5 g of an oily residue. The crude oil was purified by flash chromatography (SiO₂), eluting with a 2.5:7.5 petroleum ether-ethyl acetate mixture, to give 2.25 g of a yellow oil.

Yield: 75%

R_{f}: 0.45 (2.5:7.5 petroleum ether-ethyl acetate)

B.p.: 163° C./0.1 mmHg

IR ν (cm⁻¹): (Film) 1600 (C═C, Ar), 1670 (C═OR).

UV λ max (log ε): 208.4 (4.26)

¹H-NMR (CDCl₃): 1.72 (d, 1H, CH, J=8.4 Hz), 2.62-2.76 (m, 1H, CH), 3.13 (d, A of AB, 1H, CH, J=12.4 Hz), 3.29-3.48 (m, 1H, CH), 3.55 (d, B of AB, 1H, CH, J=13.2 Hz), 3.67-3.78 (m, 1H, CH), 4.58 (d, A of AB, 1H, CH, J=14.2 Hz), 4.78 (d, B of AB, 1H, CH, J=14.4 Hz), 7.12-7.42 (m, 10H, ArH).

Anal.: for ClgH₂ON₂O. Calc. (found) C, 78.05; (77.75); H, 6.89; (6.90); N, 9.58; (9.54).

### Example 7—3-Benzyl-3,6-diazabicyclo[3.1.1]eptan-2-one (XVIa)

An ethanol solution (51 ml) of (XVa) (3.50 g, 11.97 mmoles) was hydrogenated at 3.1×10⁵ Pa (45 psi) and 60° C. for seven hours in the presence of Pd—C 10% (1.27 g, 1.19 mmoles). The catalyst was filtered off and the solution was evaporated to give 3.0 g of a thick oil. The crude oil was purified by flash chromatography (SiO₂), eluting with a 9:1 chloroform-methanol mixture, to give 2.23 g of a waxy clear solid.

Yield: 92%

R_{f}: 0.54 (9:1 chloroform-methanol)

m.p.: 61-62° C.

IR ν (cm⁻¹): (Nujol) 1600 (C═C Ar), 1670 (C═OR).

UV λ max (log ε): 208.8 (4.13)

¹H-NMR (CDCl₃): 1.74 (d, 1H, CH, J=9.00 Hz), 2.80-2.96 (m, 1H, CH), 3.24-3.50 (m, 3H), 3.76-3.90 (m, 1H, CH), 4.51 (d, A of AB, 1H, J=14.6 Hz), 4.72 (d, B of AB, 1H, J=14.6 Hz), 7.23-7.42 (m, 5H, ArH).

Anal.: for C₁₂H₁₄N₂O. Calc. (found) C, 71.26; (70.97); H, 6.98; (6.87); N, 13.85; (13.78).

### Example 8—3-Benzyl-3,6-diazabicyclo[3.1.1]heptane (VAa)

A tetrahydrofuran solution of (XVIa) (2.16 g, 10.68 mmoles) was dropped into a suspension of lithium aluminium hydride (1.70 g, 42.72 mmoles) in tetrahydrofuran, at 0° C. The mixture was allowed to warm to room temperature, refluxed overnight, then cooled to 0° C. and added in succession with ethyl ether (49.62 ml), water (1.52 ml), 2 N NaOH (1.52 ml) and water (4.58 ml). The mixture was filtered and the filtrate was evaporated to give 1.90 g of a clear oil used in the subsequent step without further purification.

Yield: 94%

R_{f}: 0.17 (9:1 chloroform-methanol)

B.p.: 155° C./0.1 mmHg

IR ν (cm⁻¹): (Film) 1590 (C═C Ar)

UV λ (max (log ε): 206.8 (4.04)

¹H-NMR (CDCl₃): 1.93 (d, 1H, CH, J=8.0 Hz), 2.40-2.53 (m, 1H, CH), 2.65 (d, A of AB, 1H, CH, J=11.0 Hz), 3.07 (d, B of AB, 1H, CH, J=10.6 Hz), 3.51-3.62 (m, 2H), 3.72 (s, 2H, CH₂), 7.22-7.41 (m, 5H, ArH).

Anal.: for C₁₂H₁₆N₂ Calc. (found) C, 76.55; (76.23); H, 6.98; (6.95); N, 13.85; (13.80).

### Example 9—3-Benzyl-6-t-butoxycarbonyl-3,6-diazabicyclo[3.1.1]heptane (VIIa)

A solution of di-tert-butyl dicarbonate (2.36 g, 10.51 mmoles) in tetrahydrofuran (10.7 ml) was cooled to 0° C. and added with a solution of (VAa) (1.32 g, 7.01 mmoles) in tetrahydrofuran (15 ml). The mixture was left under stirring for 10 minutes, allowed to warm to room temperature and left under stirring for further 12 hours. The mixture was added with a 10% aqueous NaHCO₃ solution and extracted with ethyl ether (14 ml×2). The combined organic phases were washed with water, dried over Na₂SO₄ and concentrated to give 1.80 g of an oily residue. The residue was purified by flash chromatography (SiO₂), eluting with a 2:8 petroleum ether-ethyl acetate mixture, to give 1.71 g of a clear oil.

Yield: 85%

R_{f}: 0.52 (8:2 petroleum ether-ethyl acetate)

B.p.: 170° C./0.1 mmHg

IR ν (cm⁻¹): (Film) 1600 (C═C Ar), 1670 (C═OR)

UV λ max (log ε): 208.4 (3.84).

¹H-NMR (CDCl₃): 1.45 (s, 3H, CH₃), 1.72 (d, 1H, CH, J=8.0 Hz), 2.30-2.47 (m, 1H CH), 2.70-3.30 (m, 4H, CH₂×2), 3.69 (s, 2H, CH₂), 3.99-4.15 (m, 2H, CHx2), 7.22-7.40 (m, 5H, ArH).

Anal.: for C₁₇H₂₄N₂O₂ Calc. (found) C, 70.80; (70.55); H, 8.39; (8.36); N, 9.71; (9.67).

### Example 10⁻⁶-t-Butoxycarbonyl-3,6-diazabicyclo[3.1.1]heptane (VBa)

A solution of (VIIa) (1.49 g, 5.16 mmoles) in ethanol (15 ml) was hydrogenated at 3.1×10⁵ Pa (45 psi) and 60° C. for seven hours in the presence of 10% Pd—C (0.55 g, 0.52 mmoles). The catalyst was filtered off and the solution was evaporated to give 1.5 g of a crude oil, which was purified by flash chromatography (SiO₂), eluting with a 9:1 chloroform-methanol mixture to give 0.89 g of a clear oil.

Yield: 87%

R_{f}: 0.43 (9:1 chloroform-methanol)

B.p.: 164° C./0.1 mmHg

IR ν (cm⁻¹): (Film) 1710 (C═OR)

UV λ max (log ε): 204.9 (3.44)

¹H-NMR (CDCl₃): 1.47 (s, 3H, CH₃), 1.55 (d, 1H, CH, J=8.4 Hz), 2.49-2.68 (m, 1H, CH), 2.91 (d, 2H, CH₂, J=12.6 Hz), 3.30-3.62 (m, 2H, CH₂), 3.96-4.13 (m, 2H, CH₂).

Anal.: for C₁₀H₁₈N₂O₂ Calc. (found) C, 60.58; (60.38); H, 9.15; (9.11); N, 14.13; (14.07).

### Example 11—3-Benzyl-6-propionyl-3,6-diazabicyclo[3.1.1]heptane (VIAa)

A solution of (VAa) (2.00 g, 10.62 mmoles) in dichloromethane (45 ml), at a temperature of 0° C., was added with propionic anhydride (4.94 ml, 38.55 mmoles) dissolved in 10 ml of dichloromethane. The mixture was refluxed for one hour, then cooled to 0° C. and added with a 20% aqueous NaOH solution to alkaline pH. The mixture was left under stirring overnight at room temperature, then extracted with dichloromethane. The organic phase was dried (Na₂SO₄), and concentrated to give 2.50 g of a residue which was purified by flash chromatography (SiO₂), eluting with a 2:8 petroleum ether-ethyl acetate mixture, to give 2.10 g of a clear oil.

Yield: 82%

R_{f}: 0.22 (2:8 petroleum ether-ethyl acetate)

B.p.: 161° C./0.1 mmHg

IR ν (cm⁻¹): (Film) 1590 (C═C Ar), 1630 (C═OR).

UV λ max (log ε): 207.9 (4.14)

¹H-NMR (CDCl₃): 1.93 (d, 1H, CH, J=8.0 Hz), 2.40-2.53 (m, 1H, CH), 2.65 (d, A of AB, 1H, CH, J=11.0 Hz), 3.07 (d, B of AB, 1H, CH, J=10.6 Hz), 3.51-3.62 (m, 2H), 3.72 (s, 2H, CH₂), 7.22-7.41 (m, 5H, ArH).

Anal.: for C₁₅H₂₀N₂O Calc. (found) C, 73.74; (73.45); H, 8.25; (8.22); N, 11.47; (11.43).

### Example 12—6-Propionyl-3,6-diazabicyclo[3.1.1]heptane (IIIAa)

An ethanol solution (18 ml) of the compound of Example 11 (1.83 g, 7.49 mmoles) was hydrogenated at 3.1×10⁵ Pa (45 psi) and 60° C. for seven hours in the presence of Pd—C 10% (0.80 g, 0.075 mmoles). The catalyst was filtered and the solution was evaporated to give 2.0 g of an oily reside. The crude oil was purified by flash chromatography (SiO₂) eluting with a 9:1 chloroform-methanol mixture to give 1.09 g of a clear waxy solid.

Yield: 95%

R_{f}: 0.13 (8:2 chloroform-methanol)

m.p.: 58-60° C.

IR ν (cm⁻¹): (Nujol) 1640 (C═OR).

UV λ max (log ε): 205.7 (3.57)

¹H-NMR (CDCl₃): 1.16 (t, 3H, CH₃, J=7.4 Hz), 1.75 (d, 1H, CH, J=6.2 Hz), 2.41 (q, 2H, CH₂, J=7.4 Hz), 3.10-3.28 (m, 1H, CH), 3.82-4.19 (m, 3H), 4.37 (d, 1H, CH, J=12.2 Hz), 4.56 (d, 2H, CH₂, J=12.2 Hz).

Anal.: for C₈H₁₄N₂O Calc. (found) C, 62.31; (62.09); H, 9.15; (9.11); N, 18.17; (18.10).

### Example 13—3-Propionyl-6-t-butoxycarbonyl-3,6-diazabicyclo[3.1.1]heptane (VIBa)

A dichloromethane solution of (VBa) (0.20 g, 1.00 mmoles) was added with triethylamine (0.72 ml, 5.04 mmoles) and propionic anhydride (0.49 ml, 3.65 mmoles), cooling at 0° C. The mixture was left under stirring for three hours at room temperature, then washed with water. The organic phase was dried (Na₂SO₄), and concentrated to give 0.600 g of a dark oily residue. The crude oil was purified by flash chromatography (SiO₂), eluting with a 3:7 petroleum ether-ethyl acetate mixture, to give 0.22 g of a clear oil.

Yield: 86%

R_{f}: 0.28 (3:7 petroleum ether-ethyl acetate)

B.p.: 167° C./0.1 mmHg

IR ν (cm⁻¹): (Film) 1650 (C═OR, amide), 1700 (C═OR, ester)

UV λ max (log ε): 205.6 (3.80)

¹H-NMR (CDCl₃): 1.16 (t, 3H, CH₃, J=7.4 Hz), 1.36 (d, 1H, CH, J=8.8 Hz), 1.12 (s, 3H, CH₃), 2.32 (q, 2H, CH₂, J=7.4 Hz), 2.52-2.69 (m, 1H, CH), 3.38-3.57 (m, 2H, CH₂), 3.94-4.09 (m, 2H, CH₂), 4.11-4.24 (m, 2H, CHx2).

Anal.: for C₁₃H₂₂N₂O₂ Calc. (found) C, 61.39; (61.15); H, 8.72; (8.69); N, 11.01; (10.97).

### Example 14—3-Propionyl-3,6-diazabicyclo[3.1.1]heptane (IIIBa)

A solution of the compound of Example 13 (0.43 g, 1.69 mmoles) in dichloromethane (5.60 ml) was added with trifluoroacetic acid (2.60 ml, 54.20 mmoles) and left under stirring for 2.5 hours at 0° C. The solution was concentrated and the residue was dissolved in 20% aqueous K₂CO₃ solution. The aqueous phase was extracted with dichloromethane, dried (Na₂SO₄) and concentrated, to give 0.200 g of a waxy solid.

Yield: 77%

R_{f}: 0.22 (8:2 chloroform-methanol)

m.p.: 98-100° C. as hydrochloride

IR ν (cm⁻¹): (Nujol) 1600 (C═C, Ar), 1660 (C═OR)

UV λ max (log ε): 206.3 (3.68)

¹H-NMR (CDCl₃): 1.19 (t, 3H, CH₃, J=7.2 Hz), 1.47 (d, 1H, CH, J=9.0 Hz), 2.36 (q, 2H, CH₂, J=7.4 Hz), 2.62-2.80 (m, 1H, CH), 3.58-3.88 (m, 6H).

Anal.: for C₈H₁₄N₂O. Calc. (found) C, 62.31; (62.06); H, 9.15; (9.12); N, 18.17; (18.09).

### Example 15—General procedure for the Preparation of 3-alkyl-6-t-butoxycarbonyl-3,6-diazabicyclo[3.1.1]heptanes

A solution of (VBa) (2.52 mmoles) and of an aldehyde of formula (IV) (2.77 mmoles) in acetonitrile (20 ml), kept at 0° C., was added with sodium cyanoborohydride (3.53 mmoles) in small portions. The mixture was stirred at room temperature for 15 min and the pH was adjusted to neutrality with glacial acetic acid. The mixture was stirred for 24 hours at room temperature, then the solvent was evaporated off and the residue taken up with 10 ml of 2 N aq KOH. The aqueous phase was extracted with Et₂O, and the extracts were combined, dried over sodium sulfate and evaporated. The residue was purified by flash chromatography (7:3 petroleum ether/AcOEt) to give the compounds (VIII).

### Example 16—General Procedure for the Preparation of 3-alkyl-3,6-diazabicyclo[3.1.1]heptanes

A dichloromethane solution (10 ml) of the compounds of Example 15 (0.95 mmoles) was added with trifluoroacetic acid (19.08 mmoles) and left under stirring at room temperature for 12 hours. The solution was concentrated and the residue was dissolved in a 20% aqueous K₂CO₃ solution. The aqueous phase was extracted with dichloromethane, dried (Na₂SO₄) and concentrated, to give the desired products (IX).

### Example 17—General Procedure for the Preparation of 3-propionyl-6-alkyl-3,6-diazabicyclo[3.1.1]heptanes

An acetonitrile solution (7 ml) of (IIIA) or (IIIB) (0.97 mmoles) and of an aldehyde (IVa) (1.07 mmoles), kept at 0° C., was added with sodium cyanoborohydride (1.36 mmoles) in small portions. The mixture was kept under stirring at room temperature for 15 min and the pH was adjusted to neutrality with glacial acetic acid. The mixture was stirred for 24 hours at room temperature, then the solvent was evaporated off and the residue taken up with 10 ml of 2 N aq KOH. The aqueous phase was extracted with Et₂O, then the extracts were combined, dried over sodium sulfate and evaporated. The residue was purified by flash chromatography (acetone/dichloromethane 8:2) to give the compounds (IA) as oils (see table 1).

### Example 18—General Procedure for the Preparation of 3-alkyl-6-propionyl-3,6-diazabicyclo[3.1.1]heptanes

A dichloromethane solution (6 ml) of (IX) (0.28 mmoles), kept at 0° C., was added with propionic anhydride (0.98 mmoles) dissolved in 2 ml of dichloromethane. The mixture was refluxed for one hour, then cooled to 0° C. and added with a 20% NaOH aqueous solution to alkaline pH. The mixture was left under stirring overnight at room temperature, then extracted with dichloromethane. The organic phase was dried (Na₂SO₄), and concentrated to give an oily residue that was purified by flash chromatography (SiO₂), eluting with a 6:4 petroleum ether-ethyl acetate mixture, to give the compounds (IB) as oils (see table 1).

TABLE 1

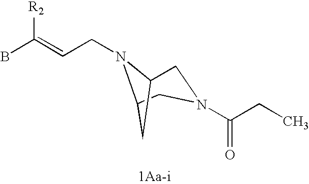

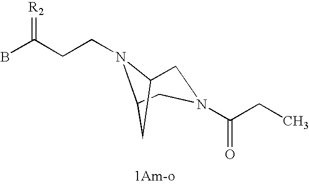

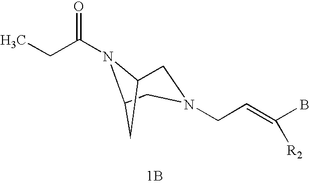

| | | | Yeld | m.p.^{a} | Formula | IR | ¹H-NMR |
|---|---|---|---|---|---|---|---|
| Example | R₂ | B | % | ° C. | (analysis^{b}) | ν cm⁻¹ | δ ppm |
| (IAa) | H | Ph | 79 | 142-144 | C₁₇H₂₂N₂O | 1660 | 1.21 (t, 3H, CH₃, J=7.4 Hz), 1.50 (d, 1H, CH, J=8.8 Hz), 2.38 |
| | | | | | (C,H,N) | | (q, 2H, CH_{2.} J=7.4 Hz), 2.68-2.78 (m, 1H, CH), 3.24 (d, 2H, |
| | | | | | | | CH₂, J=6.2 Hz), 3.38-3.89 (m, 6H), 6.19 (dt, 1H, CH, J₁=6.2 Hz, |
| | | | | | | | J₂=16.0 Hz), 6.56 (d, 1H, CH, J=16.0 Hz), 7.20-7.48 (m, 5H, ArH) |
| (IAb) | H | 2-Cl-Ph | 34 | 137-139 | C₁₇H₂₁ClN₂O | 1660 | 1.21 (t, 3H, CH₃, J=7.8 Hz), 1.52 (d, 1H, CH, J=8.8 Hz), 2.38 |
| | | | | | (C,H,N) | | (q, 2H, CH₂, J=7.6 Hz), 2.59-2.78 (m, 1H, CH), 3.28 (d, 2H, CH₂, |
| | | | | | | | J=5.8 Hz), 3.40-3.88 (m, 6H), 6.17 (dt, 1H, CH, J₁=9.8 Hz, |
| | | | | | | | J₂=15.8 Hz), 6.94 (d, 1H, CH, J=15.6 Hz), 7.27-7.59 (m, 4H, ArH) |
| (IAc) | H | 3-Cl-Ph | 41 | 144-146 | C₁₇H₂₁ClN₂O | 1650 | 1.21 (t, 3H, CH₃, J=7.4 Hz), 1.50 (d, 1H, CH, J=8.8 Hz), 2.37 (q, |
| | | | | | (C,H,N) | | 2H, CH₂, J=7.4 Hz), 2.58-2.72 (m, 1H, CH), 3.22 (d, 2H, CH₂, |
| | | | | | | | J=6.0 Hz), 3.40-3.84 (m, 6H), 6.10-6.30 (m, 1H, CH), 6.50 (d, 1H, CH, |
| | | | | | | | J=15.2 Hz), 7.18-7.31 (m, 3H, ArH), 7.33 (s, 1H, ArH) |
| (IAd) | H | 4-Cl-Ph | 35 | 142-143 | C₁₇H₂₁ClN₂O | 1660 | 1.21 (t, 3H, CH₃, J=7.6 Hz), 1.50 (d, 1H, CH, J=8.8 Hz), 2.38 (q, 2H, |
| | | | | | (C,H,N) | | CH₂, J=7.6 Hz), 2.59-2.78 (m, 1H, CH), 3.27 (d, 2H, CH₂, J=5.8 Hz), |
| | | | | | | | 3.41-3.89 (m, 6H), 6.16 (dt, 1H, CH, J₁=6.2 e J₂=15.8 Hz), |
| | | | | | | | 6.94 (d, 1H, CH, J=15.6 Hz), 7.18-7.31 (m, 4H, ArH) |
| (IAe) | CH₃ | Ph | 51 | 123-125 | C₁₈H₂₄N₂O | 1670 | 1.21 (t, 3H, CH₃, J=7.4 Hz), 1.51 (d, 1H, CH, J=8.0 Hz), 2.05 |
| | | | | | (C,H,N) | | (s, 3H, CH₃), 2.37 (q, 2H, CH₂, J=7.0 Hz), 2.57-2.73 (m, 1H, CH), |
| | | | | | | | 3.26 (d, 2H, CH₂, J=6.0 Hz), 3.37-3.83 (m, 6H), 5.68-5.80 |
| | | | | | | | (m, 1H, CH), 7.19-7.44 (m, 5H, ArH) |
| (IAf) | CH₃ | 4-Cl-Ph | 65 | 119-121 | C₁₈H₂₃ClN₂O | 1650 | 1.20 (t, 3H, CH₃, J=7.4 Hz), 1.51 (d, 1H, CH, J=8.8 Hz), 2.02 (s, |
| | | | | | (C,H,N) | | 3H, CH₃), 2.37 (q, 2H, CH₂, J=7.0 Hz), 2.58-2.76 (m, 1H, CH), 3.24 |
| | | | | | | | (d, 2H, CH₂, J=6.0 Hz), 3.41-3.88 (m, 6H), 5.67-5.80 (m, 1H, CH), |
| | | | | | | | 7.22-7.40 (m, 4H, ArH) |
| (IAg) | CH₃ | 3,4-Cl₂-Ph | 29 | 122-124 | C₁₈H₂₂Cl₂N₂O | 1630 | 1.21 (t, 3H, CH₃, J=7.4 Hz), 1.52 (d, 1H, CH, J=9.0 Hz), 2.02 |
| | | | | | (C,H,N) | | (s, 3H, CH₃), 2.37 (q, 2H, CH₂, J=7.8 Hz), 2.58-2.75 (m, 1H, CH), |
| | | | | | | | 3.24 (d, 2H, CH₂, J=6.4 Hz), 3.43-3.89 (m, 6H), 5.70-5.85 |
| | | | | | | | (m, 1H, CH), 7.15-7.52 (m, 4H, ArH) |
| (IAh) | Et | Ph | 76 | 160-163 | C₁₉H₂₆N₂O | 1650 | 0.97 (t, 3H, CH₃, J=7.2 Hz), 1.21 (t, 3H, CH₃, J=7.6 Hz), 1.51 |
| | | | | | (C,H,N) | | (d, 1H, CH), 2.38 (q, 2H, CH₂, J=7.4 Hz), 2.51 (q, 2H, CH₂, J=7.4 Hz), |
| | | | | | | | 2.60-2.78 (m, 1H, CH), 3.27 (d, 2H, CH₂, J=6.2 Hz), 3.40-3.91 (m, |
| | | | | | | | 6H), 5.60 (t, 1H, CH, J=6.4 Hz), 7.22-7.51 (m, 4H, ArH) |
| (IAi) | H | 4-NO₂-Ph | 20 | >250 dec | C₁₇H₂₁N₃O₃ | 1650 | 1.21 (t, 3H, J=7.0 Hz), 1.51-1.56 (m, 1H), 2.38 (q, 2H, J=7.2 Hz), |
| | | | | | (C,H,N) | | 2.60-2.66 (m, 1H), 3.28 (d, 2H, J=6.2 Hz), 3.66-3.82 (m, 6H), 6.40 |
| | | | | | | | (dt, 1H, J=6.66 and 15.8 Hz), 6.64 (d, 1H, J=15.8 Hz), |
| | | | | | | | 7.46-8.20 (m, 4H, ArH) |
| (IAm) | H₂ | 4-NO₂-Ph | 40 | 140 dec | C₁₇H₂₃N₃O₃ | 1650 | 1.15-1.20 (m, 5H, J=7.0 Hz), 1.40-1.50 (m, 1H, CH₂), 1.60-1.80 (m, |
| | | | | | (C,H,N) | | 2H, CH₂), 2.30 (t, 2H, CH₂), 2.40 (q, 2H, CH₂, J=7.0 Hz), |
| | | | | | | | 2.65-2.80 (m, 1H, CH), 3.40-3.70 (m, 6H), 7.32-8.17 (m, 4H, ArH). |
| (IAn) | H₂ | 4-Cl-Ph | 25 | 144 | C₁₇H₂₃ClN₂O | 1660 | 1.15 (t, 3H, CH₃, J=7.4 Hz), 1.20-1.25 (m, 2H, CH₂), 1.40 (d, 1H, |
| | | | | | (C,H,N) | | CH, J=8.8 Hz), 1.5-1.7 (m, 2H, CH₂), 2.20-2.40 (m, 4H, CH₂, |
| | | | | | | | J=7.4 Hz), 2.58-2.70 (m, 1H, CH), 3.37-3.70 (m, 6H), 7.10-7.30 |
| | | | | | | | (m, 4H, ArH). |
| (IAo) | O | Ph | 50 | | C₁₇H₂₂N₂O₂ | | 1.10-1.18 (m, 5H, J=7.2 Hz), 1.41-1.49 (d, 1H, CH), 2.30 (q, 2H, CH₂, |
| | | | | | (C,H,N) | | J=7.2 Hz), 2.75-2.83 (m, 2H, CH₂), 2.90-3.15 (m, 1H, CH), 3.37-3.82 |
| | | | | | | | (m, 6H), 7.20-7.90 (m, 5H, ArH). |
| (IBa) | H | Ph | 75 | | C₁₇H₂₂N₂O | 1655 | 1.14 (t, 3H, CH₃, J=7.6 Hz), 2.01-2.60 (m, 5H), 2.75-3.15 (m, 3H), |
| | | | | | (C,H,N) | | 3.32 (d, 2H, CH₂, J=6.6 Hz), 6.21 (dt, 1H, CH, J=15.6 and 6.6 Hz), |
| | | | | | | | 6.54 (d, 1H, CH, J=15.6 Hz), 7.15-7.40 (m, 4H, ArH) |

^{a}IAa-h as hydrochloride. IAi, IAm-o as fumarate.
^{b}the compounds′ analysis are satisfactory and fall within the ±0.4% range of the calculated values.

Pharmacologic Activity

Compounds (IAa-o) were subjected to binding studies for opioid receptors in mouse brain homogenate, using [³H]-DAMGO for μ receptors, [³H]-Deltorphina for δ receptors, and [³H]-U69593 for κ receptors and morphine as the reference compound. The results of the binding experiments are shown in table 2.

TABLE 2
(IAa-o Affinity [Kᵢ (nM)^{a}] towards opiod receptors

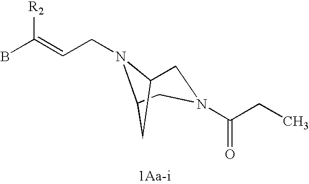

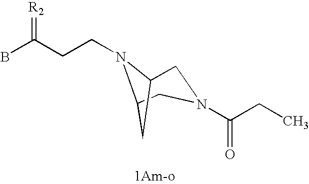

| Compound | B | R₂ | μ | δ | κ |
|---|---|---|---|---|---|
| (IAa) | Ph | H | 208 ± 8 | >5000 | >5000 |
| (IAb) | 2-Cl-Ph | H | 92 ± 4 | >5000 | >5000 |
| (IAc) | 3-Cl-Ph | H | 21 ± 0.7 | 2060 ± 70 | >5000 |
| (IAd) | 4-Cl-Ph | H | 16 ± 2 | 4100 ± 50 | >5000 |
| (IAe) | Ph | CH₃ | 178 ± 11 | >5000 | >5000 |
| (IAf) | 4-Cl-Ph | CH₃ | 7.9 ± 0.7 | 2050 ± 50 | >5000 |
| (IAg) | 3,4-Cl₂-Ph | CH₃ | 2.7 ± 0.5 | 2200 ± 200 | >5000 |
| (IAh) | Ph | Et | 384 ± 12 | >5000 | >5000 |
| (IAi) | 4-NO₂-Ph | H | 5.2 | >5000 | >5000 |
| (IAm) | 4-NO₂-Ph | H₂ | 20 ± 3 | >5000 | >5000 |
| (IAn) | 4-Cl-Ph | H₂ | 32 ± 5 | >5000 | >5000 |
| (IAo) | Ph | O | 400 ± 60 | >5000 | >5000 |
| Morphine | | | 2.5 ± 0.8 | 100.2 ± 5.14 | 280.6 ± 9.2 |

^{a}Each value is the mean ± SEM of independent experiments, each carried out in triplicate (n=3).

1. Compounds of general formula (I) 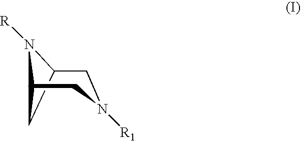
wherein R and R₁, different from one another, are:
a straight or branched C₂-C₈ acyl group; or
a group selected from: 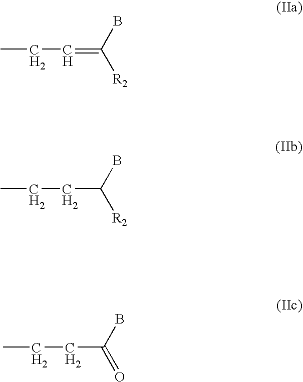
wherein B is:
a C₆-C₁₀ aryl group, optionally substituted with one or more groups, which can be the same or different, selected from the group consisting of C₁-C₃ alkoxy, C₁-C₂ halo alkyl, C₁-C₃ alkyl, halogens, carboxy, cyano, nitro, CONHR₃, wherein R₃ is straight or branched C₁-C₄ alkyl;
a C₅-C₇ cycloalkyl group;
a 5- or 6-membered aromatic heterocycle, optionally benzofused, having at least one heteroatom selected from nitrogen, oxygen, sulfur; said heterocycle optionally bearing one or more substituents selected from those indicated for the aryl group;
and in which R₂ is hydrogen, straight or branched C₁-C₄ alkyl, a C₅-C₇ cycloalkyl group or phenyl optionally substituted with one or more groups, which can be the same or different, selected from those indicated above for the aryl group;
and pharmaceutically acceptable salts thereof.

2. Compounds as claimed in claim 1 wherein the C₂-C₈ group is selected from acetyl, propionyl, butyryl, isobutyryl, valeryl, isovaleryl, pivaloyl, caproyl.

3. Compounds as claimed in claim 2 in which the C₂-C₈ group is acetyl or propionyl.

4. Compound as claimed in claim 3 in which the C₂-C₈ group is propionyl.

5. Compounds according to claim 1 in which B is an aromatic heterocyclic ring selected from pyrrole, furan, thiophene, imidazole, oxazole, thiazole, pyridine, pyrimidine, pyridazine, pyrazine, benzothienyl.

6. Compounds according to claim 1 in which B is phenyl.

7. Compounds as claimed in claim 6 in which B is phenyl substituted with one or more chlorine atoms.

8. Compounds according to claim 1 in which R₂ is selected from hydrogen and straight or branched C₁-C₄ alkyl.

9. Compounds as claimed in claim 8 in which R₂ is selected from hydrogen, methyl or ethyl.

10. A compound selected from:
3-propionyl-6-cinnamyl-3,6-diazabicyclo[3.1.1]heptane;
3-propionyl-6-[(2E)-3-(2′-chlorophenyl)-prop-2-enyl]-3,6-diazabicyclo[3.1.1]heptane;
3-propionyl-6-[(2E)-3-(3′-chlorophenyl)-prop-2-enyl]-3,6-diazabicyclo[3.1.1]heptane;
3-propionyl-6-[(2E)-3-(4′-chlorophenyl)-prop-2-enyl]-3,6-diazabicyclo[3.1.1]heptane;
3-propionyl-6-[(2E)-3-phenyl-but-2-enyl]-3,6-diazabicyclo[3.1.1]heptane;
3-propionyl-6-[(2E)-3-(4′-chlorophenyl)-but-2-enyl]-3,6-diazabicyclo[3.1.1]heptane;
3-propionyl-6-[(2E)-3-(3′,4′-dichlorophenyl)-but-2-enyl]-3,6-diazabicyclo[3.1.1]heptane;
3-propionyl-6-[(2E)-3-phenyl-pent-2-phenyl]-3,6-diazabicyclo[3.1.1]heptane;
3-propionyl-6-[(2E)-3-(4′-nitrophenyl)-prop-2-enyl]-3,6-diazabicyclo[3.1.1]heptane;
3-propionyl-6-(3-phenylpropyl)-3,6-diazabicyclo[3.1.1]heptane;
3-propionyl-6-[3-(4′-nitrophenyl)propyl)]-3,6-diazabicyclo[3.1.1]heptane;
3-propionyl-6-[3-(4′-chlorophenyl)propyl)]-3,6-diazabicyclo[3.1.1]heptane;
3-propionyl-6-(3-phenylpropyl-3-one)-3,6-diazabicyclo[3.1.1]heptane;
3-propionyl-6-[3-(4′-chlorophenyl)propyl-3-one]-3,6-diazabicyclo[3.1.1]heptane;
3-cinnamyl-6-propionyl-3,6-diazabicyclo[3.1.1]heptane.

11. Compounds according to claim 1 as central analgesics.

12. Use of the compounds according to claim 1 for the preparation of analgesic medicaments.

13. Pharmaceutical compositions containing the compounds of claim 1 in admixture with suitable excipients and/or carriers.

14. Compounds according to claim 2 in which B is an aromatic heterocyclic ring selected from pyrrole, furan, thiophene, imidazole, oxazole, thiazole, pyridine, pyrimidine, pyridazine, pyrazine, benzothienyl.

15. Compounds according to claim 3 in which B is an aromatic heterocyclic ring selected from pyrrole, furan, thiophene, imidazole, oxazole, thiazole, pyridine, pyrimidine, pyridazine, pyrazine, benzothienyl.

16. Compounds according to claim 4 in which B is an aromatic heterocyclic ring selected from pyrrole, furan, thiophene, imidazole, oxazole, thiazole, pyridine, pyrimidine, pyridazine, pyrazine, benzothienyl.

17. Compounds according to claim 2 in which B is phenyl.

18. Compounds according to claim 3 in which B is phenyl.

19. Compounds according to claim 4 in which B is phenyl.

20. Compounds according to claim 2 in which R₂ is selected from hydrogen and straight or branched C₁-C₄ alkyl.
